(19) United States
(12) Patent Application Publication
(10) Pub. No.: US 2020/0155720 A1
(43) Pub. Date: May 21, 2020

(54) MICRO-CHANNEL CONNECTORS FOR MEDICAL DEVICE DECONTAMINATION SYSTEM

(71) Applicant: Medivators Inc., Minneapolis, MN (US)

(72) Inventors: Yanhua Li, Anoka, MN (US); Adam J. Schleper, Delano, MN (US); Michael Jay Fuad, San Francisco, CA (US)

(21) Appl. No.: 16/676,699

(22) Filed: Nov. 7, 2019

Related U.S. Application Data
(60) Provisional application No. 62/769,160, filed on Nov. 19, 2018.

(51) Int. Cl. A61L 2/26; A61L 2/22; A61L 2/18; A61L 2/20

(52) U.S. Cl. CPC A61L 2/26; A61L 2/22; A61L 2/186; A61L 2202/24; A61L 2202/122; A61L 2202/123; A61L 2202/121; A61L 2/208

(57) ABSTRACT

A decontamination system for a lumen device is provided. The decontamination system comprises a lumen device container and a sterilant fluid delivery device. The lumen device container defines a lumen device receiving area that includes a fluid connector in fluid communication with the lumen device receiving area. The sterilant fluid delivery device is configured to be in fluid communication with the fluid connector. In some embodiments, the fluid connector is configured to form a loose connection with a port of a lumen device to deliver sterilant fluid into the port and leak sterilant fluid onto an external surface of the port.

### PRIORITY CLAIM

This application claims priority to and benefit of U.S. Provisional Application with Ser. No. 62/769,160 filed Nov. 19, 2018, entitled MICRO-CHANNEL CONNECTORS FOR MEDICAL DEVICE DECONTAMINATION SYSTEM, which is herein incorporated by reference in its entirety.

### TECHNICAL FIELD

This disclosure relates generally to decontamination of medical devices; in particular, this disclosure relates to a decontamination system with connector(s) that establish a loose connection with port(s) of a lumen device so sterilant fluid is introduced both inside the lumen device and on external surfaces of the port(s).

### BACKGROUND

Robust medical instruments are often sterilized at high temperatures. Commonly, the instruments are sterilized in a steam autoclave under a combination of high temperature and pressure. While such sterilization methods are very effective for more durable medical instruments, advanced medical instruments formed of rubber and plastic components with adhesives are delicate and wholly unsuited to the high temperatures and pressures associated with a conventional steam autoclave. Steam autoclaves have also been modified to operate under low pressure cycling programs to increase the rate of steam penetration into the medical devices or associated packages of medical devices undergoing sterilization. Steam sterilization using gravity, high pressure or pre-vacuum create an environment where rapid changes in temperature can take place. In particular, highly complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

Endoscopes can also present problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes.

Existing endoscope decontamination systems, also called reprocessors, include a sterilant delivery system that delivers sterilant fluid to the endoscope being reprocessed. Decontamination systems include a hookup system with connector(s) for fluidly connecting the sterilant delivery system to the endoscope. One challenge with existing sealed connectors is sterilizing the mating areas between the scope ports and the connectors. Due to the contact between the sealed connectors and scope port, the sealing areas are not sterilized.

Therefore, a need exists that overcomes one or more of the disadvantages of present decontamination systems.

### SUMMARY OF THE INVENTION

According to one aspect, this disclosure provides a decontamination system for a lumen device. The decontamination system comprises a lumen device container and a sterilant fluid delivery device. The lumen device container defines a lumen device receiving area that includes a fluid connector in fluid communication with the lumen device receiving area. The sterilant fluid delivery device is configured to be in fluid communication with the fluid connector. In some embodiments, the fluid connector is configured to form a loose connection with a port of a lumen device to deliver sterilant fluid into the port and leak sterilant fluid onto an external surface of the port.

According to another aspect, this disclosure provides a connector for connecting a lumen device to a source of sterilant fluid in an endoscope reprocessor. The connection comprises a connector body including an inlet configured to be in fluid communication with a sterilant fluid delivery device and an outlet configured to be in fluid communication with a port of a lumen device. In some embodiments, the connector body is configured to form a loose connection with the port of the lumen device to deliver sterilant fluid into the port and leak sterilant fluid onto an external surface of the port.

According to a further aspect, this disclosure provides a method of reprocessing an endoscope. The method includes the step of coupling a fluid connector of an endoscope reprocessor with a port of a lumen device. The fluid connector is configured to form a loose connection with the port such that the loose connection includes one or more gaps between the fluid connector and the port through which sterilant fluid is configured to flow. The method includes the step of introducing sterilant fluid into the port through the fluid connector. The sterilant fluid is introduced onto the external surface of the port through the gap between the fluid connector and the port.

### BRIEF DESCRIPTION OF THE DRAWINGS

The present disclosure will be described hereafter with reference to the attached drawings which are given as non-limiting examples only, in which:

FIG. 1 is diagrammatic view of a system for decontaminating a medical device according to an embodiment of the present disclosure;

FIG. 2 is a side view of an example connector according to a first embodiment of the present disclosure;

FIG. 3 is a side view of an example connector according to a second embodiment of the present disclosure;

FIG. 4 is a side view of an example connector according to a third embodiment of the present disclosure;

FIG. 5 is a side cross-sectional view of the example connector shown in FIG. 3;

FIG. 6 is a side cross-sectional view of the example connector shown in FIG. 4;

FIG. 7 is a side cross-sectional view of the example connector shown in FIG. 2;

FIG. 8 is a perspective view of an example connector according to a fourth embodiment of the present disclosure; and

FIG. 9 is a side cross-sectional view of the example connector shown in FIG. 8.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

This disclosure relates to connectors for delivery of sterilant fluid in a decontamination system. In some embodiments, this system uses connector(s) that provide a “loose” or “leaky” connection with port(s) of a lumen device to introduce sterilant fluid inside and on external surfaces outside and around the lumen device's ports. In some embodiments, the connection is “loose” or “leaky” because there are gap(s) between the port and the connector, which allow sterilant fluid to flow onto external surfaces of the port. In other embodiments, the connector is at least partially porous to introduce sterilant fluid inside and on external surfaces of the lumen device's port. By introducing flow both inside and on external surfaces of the lumen device's ports, the connectors achieve a higher level of decontamination for the lumen device.

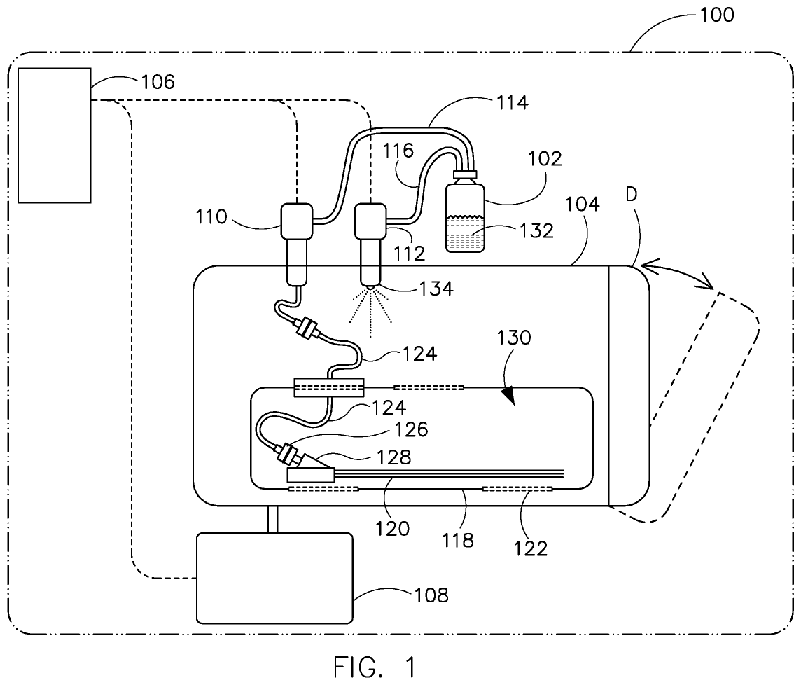

FIG. 1 is a diagrammatic view of one embodiment of a system 100 for decontaminating a medical, dental, or other device having one or more lumens extending therethrough. The system includes a reservoir 102, a decontamination chamber 104, a system controller 106, an environmental monitoring and control system 108, and vaporizers 110 and 112 which are connected to the reservoir 102 by conduits 114 and 116. A lumen device container 118 containing a lumen device 120 for decontamination may be placed within the decontamination chamber 104. In the illustrated embodiment, the container 118 can include a plurality of openings or pores 122. The reservoir 102 may be in fluid communication with the decontamination chamber 104 via vaporizer 112. The reservoir 102 may also be in fluid communication with one or more lumens extending through the lumen device 120 via vaporizer 110 and fluid conduit 124. In the embodiment shown, a connector 126 fluidly connects the fluid conduit 124 to a port 128 of the lumen device 120. In some embodiments, a plurality of connectors could connect with a plurality of ports on the lumen device, such as through a hookup system with a plurality of connectors arranged to connect with ports on the lumen device.

The system controller 106 provides control signals to and/or receives condition sensing and equipment status signals from the reservoir 102, the decontamination chamber 104, environmental monitoring and control system 108, and/or the vaporizers 110, 112. In some embodiments, the system 100 can be assembled in a device small enough to sit on a tabletop or counter. For example, the decontamination chamber 104 may have an interior volume of less than about ten cubic feet.

The lumen device 120 to be decontaminated can be placed into the decontamination chamber 104 by opening the door D and placing the lumen device 120 on a rack or other supporting assembly in the interior of the decontamination chamber 104. In some embodiments, the lumen device 120 may be enclosed in the container 118 before being placed in the decontamination chamber 104. In the example shown, the container 118 defines a lumen device receiving area 130 to receive the lumen device 120 for decontamination. In the illustrated embodiment, the container 118 includes a plurality of openings or pores 122.

The reservoir 102 may be a holding tank or other assembly configured to hold a sterilant fluid 132. In some embodiments, the sterilant fluid 132 can be a chemical or other substance suitable for use in a sterilization process that complies with the International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). In some embodiments, the sterilant fluid 132 can be a room temperature (e.g., 20.degree. C. to 25.degree. C.) substance that can be dispersed as a fluid, such as a liquid, a vapor, or a combination thereof (such as a fog) during the decontamination process. Suitable substances for the sterilant fluid 132 include hydrogen peroxide (H.sub.20.sub.2) and peracetic acid (PAA).

The container 118 is sized so that the lumen device 120 to be decontaminated fits within the container 118. In some embodiments, the container 118 may be generally described as having a top, a bottom, and four sides extending between the top and bottom to create a cube-like structure. However, the container 118 may have any suitable shape which encloses the lumen device 120. In some embodiments, the container 118 may be formed from a rigid material such that the container 118 has a rigid or structured shape. Alternatively, the container 118 may be formed from a flexible material such that the container 118 has a flexible shape. In some embodiments, the container 118 may be a terminal package. Suitable materials for the container 118 include but are not limited to a polymeric non-woven sheet, such as spun-bonded polyethylene (e.g., Tyvek®, sold by E.I. du Pont de Nemours and Company, Wilmington, Del.), and polymeric materials such as polyester and polypropylene. Suitable materials for container 118 having a rigid or structured shape include but are not limited to various metals such as aluminum, stainless steel and/or various polymers in rigid form such as polyethylene and/or polypropylene.

The lumen device 120 may be positioned within the container 118 and subjected to one or more decontamination cycles. Suitable lumen devices include any medical, dental or other device having at least one lumen extending through at least a portion of the device. In some embodiments, the lumen device 120 may include at least one lumen extending the entire length of the device. For example, the lumen device 120 may be an endoscope.

The container 118 may be configured to prevent or reduce microbes and/or other contaminants from entering the container 118. In some embodiments, for example, the container 118 can include a material suitable for allowing flow of a sterilant fluid, such as hydrogen peroxide (H₂O₂) and/or peracetic acid (PAA), into the lumen device receiving area 130 of the container 118 and blocking or reducing the flow of contaminants into the interior of the container 118. In the illustrated embodiment, the container 118 includes a plurality of openings or pores 122 for allowing flow of the sterilant fluid 132 into the container 118. In some embodiments, the pores 122 may be sized so as to allow the sterilant fluid 132 and/or air to communicate into and out of the container 118 as well as prevent microbes from entering the container 118.

In some embodiments, the sterilant fluid 132 can flow concurrently from the reservoir 102 to vaporizers 110, 112 and subsequently to decontamination chamber 104 and lumen device 120. In other embodiments, the flow of the sterilant fluid 132 to vaporizer 110 may initiate before or after the initiation of flow of the sterilant fluid 132 to vaporizer 112. The sterilant fluid 132 from vaporizer 110 may decontaminate the internal and external surfaces of the lumen device's 120 port 128 via the connector 126 and the sterilant fluid 132 from the vaporizer 112 may decontaminate the exterior surfaces of lumen device 120 as well as the surfaces of the container 118. As explained below, the connector 126 aids in decontaminating exterior surfaces of lumen device's 120 port 128 due to a “loose” or “leaky” connection that allows the sterilant fluid 132 to flow over the external surfaces of the port 128. The amount of sterilant fluid 132 introduced into the decontamination chamber 104, the lumen device 120 or a combination thereof can be controlled by the system controller 106 by controlling the amount of the sterilant fluid 132 fed or delivered to vaporizers 110, 112. The rate and amount of the sterilant fluid 132 delivered to vaporizers 110, 112 may be preprogrammed into the system controller 106 or may be manually entered into the system controller 106 by a user of the system 100.

To decontaminate a lumen device, such as a medical, dental or other device, the lumen device 120 may be sealed within the container 118 and placed in the decontamination chamber 104. The lumen device 120 is then subjected to a decontamination process which may include one or more decontamination cycles. A suitable cycle may include adjusting the pressure of the decontamination chamber 104 to a suitable range, such as to a pressure less than 10 Torr, conditioning using plasma, and introducing the sterilant fluid 132 into the decontamination chamber 104 via vaporizer 112 and nozzle 134 and introducing the sterilant fluid 132 into and on exterior port surfaces of the lumen device 120 via the vaporizer 110, conduit 124, and connector 126. The sterilant fluid 132 may be held within the decontamination chamber 104 for a period of time to facilitate the decontamination of the lumen device 120, and in particular, the exterior surfaces of the lumen device 120. Similarly, the sterilant fluid 132 may be held within the lumen device 120 for a period of time to facilitate the decontamination of the interior surfaces or lumen(s) of the lumen device 120. When the sterilant fluid 132 has been held in the decontamination chamber 104 for the desired or programmed amount of time, the system controller 106 can vent the decontamination chamber 104 to a higher, but sub-atmospheric pressure. The system controller 106 can then hold the pressure within the decontamination chamber 104 for a period of time to further facilitate the decontamination of the load. Following the hold period, the system controller 106 may evacuate the decontamination chamber 104 to remove the sterilant fluid residuals from the decontamination chamber 104 which may also include a plasma treatment to further enhance the removal of the substance residuals, followed by venting the decontamination chamber 104. This cycle or steps may be repeated or extended as part of a comprehensive cycle.

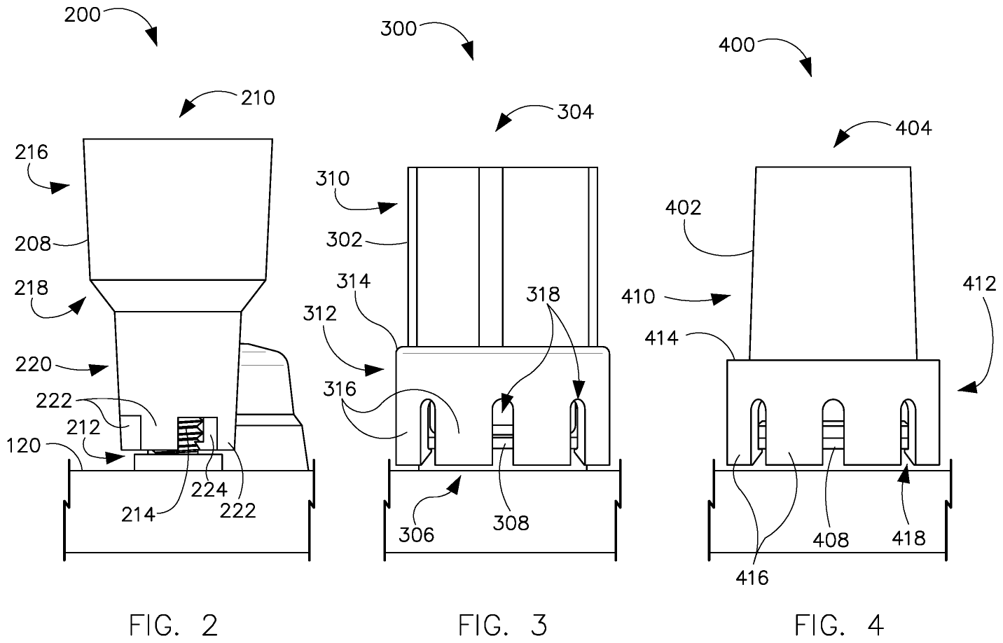

FIGS. 2-4 illustrate side views of connectors according to various embodiments of this disclosure formed from a non-porous material. FIG. 2 illustrates a connector 200 according to a first embodiment. FIG. 3 illustrates a connector 300 according to a second embodiment. FIG. 4 illustrates a connector 400 according to a third embodiment. In some embodiments, the connectors 200, 300, 400 may be configured for fluid connection with various types of ports on a hookup system and for connection with different types of ports on the lumen device 120. Although the configurations of the connectors 200, 300, 400 are shown for illustrative purposes, the connectors 200, 300, 400 could have other configurations depending on the ports to which the connectors 200, 300, 400 are to connect. In some embodiments, the connectors 200, 300, 400 could be formed as part of a hookup system; however, in some embodiments the connectors 200, 300, 400 may be arranged separately in the system 100 for connection with port(s) 128 of the lumen device 120 depending on the circumstances.

In the embodiments shown in FIGS. 2-4, the connectors 200, 300, 400 establish a “loose” or “leaky” connection with ports of the lumen device 120 to introduce sterilant fluid both within the lumen(s) of the lumen device 120 and on external surfaces of the lumen device's 120 port 128. Unlike the “sealed” connection in existing connectors, a loose or leaky connection leaks sterilant fluid on external surfaces of the port as sterilant fluid flows into the lumen(s). This is an intentional leak that introduces sterilant fluid on external surfaces of the port that would otherwise be blocked with existing sealed connectors. In some embodiments, the connectors 200, 300, 400 include microchannels and gaps between the connector and the corresponding port of the lumen device to create a balance of flow inside the lumen device's lumens and flow through all the connecting areas to achieve total decontamination of the entire lumen device, including inside the lumens and all areas outside and around the lumen device's ports.

Depending on the circumstances, the connectors 200, 300, 400 could be configured for a single use. For example, in some embodiments, the connectors 200, 300, 400 could be formed from polypropylene (PP) or other suitable polymer material. With such a material, the connectors 200, 300, 400 can be manufactured with a sufficiently low cost to make single use feasible. In such embodiments, there is no need to worry about sterilizing the connectors 200, 300, 400 for reuse, which eliminates the risk of cross-contamination.

In the embodiment shown in FIG. 2, the connector 200 includes a body 208 with a first end 210 and opposing second end 212. In the embodiment shown, the first end 210 is configured to be connected with a sterilant fluid delivery device, such as a hookup system, to push or pull sterilant fluid into/out of the lumen device 120. The second end 212 is configured to establish a “leaky” connection with a first port 214 of the lumen device 120 in the example shown. As shown, the body 208 of the connector 200 includes a first section 216 extending from the first end 210 to an angled section 218. A smaller diameter section 220 extends between the angled section 218 and the second end 212 in this example. As shown, the smaller diameter section 220 includes a plurality of latching projections 222 configured to form a leaky connection with the first port 214. In this embodiment, the latching projections 222 are arranged in a ring-like shape. As shown, the body 208 includes a plurality of gaps 224 between the latching projections 222. In the example shown, the body 208 is generally tapered between the first end 210 and the second end 212.

In the embodiment shown in FIG. 3, the connector 300 includes a body 302 with a first end 304 and an opposing second end 306. In the embodiment shown, the first end 304 is configured to be connected with a sterilant fluid delivery device, such as a hookup system, to push or pull sterilant fluid into/out of the lumen device 120. The second end 306 is configured to establish a “leaky” connection with a second port 308 of the lumen device 120. As shown, the body 302 of the connector 300 includes a first section 310 extending from the first end 304 to a second section 312. In the embodiment shown, the second section 312 has a larger diameter than the first section 310, with a shoulder 314 transitioning between the sections 310, 312. As shown, the second section 312 includes a plurality of latching projections 316 configured to form a leaky connection with the second port 308. In this embodiment, the latching projections 316 are arranged in a ring-like shape. As shown, the body 302 includes a plurality of gaps 318 between the latching projections 316.

In the embodiment shown in FIG. 4, the connector 400 includes a body 402 with a first end 404 and an opposing second end 406. In the embodiment shown, the first end 404 is configured to be connected with a sterilant fluid delivery device, such as a hookup system, to push or pull sterilant fluid into/out of the lumen device 120. The second end 406 is configured to establish a “leaky” connection with a third port 408 of the lumen device 120. As shown, the body 402 of the connector 400 includes a first section 410 extending from the first end 404 to a second section 412. In the embodiment shown, the second section 412 has a larger diameter than the first section 410, with a shoulder 414 transitioning between the sections 410, 412. As shown, the second section 412 includes a plurality of latching projections 416 configured to form a leaky connection with the third port 408. In this embodiment, the latching projections 416 are arranged in a ring-like shape. As shown, the body 402 includes a plurality of gaps 418 between the latching projections 416. In the embodiment shown, the first section 410 generally tapers from the shoulder 414 towards the first end 404.

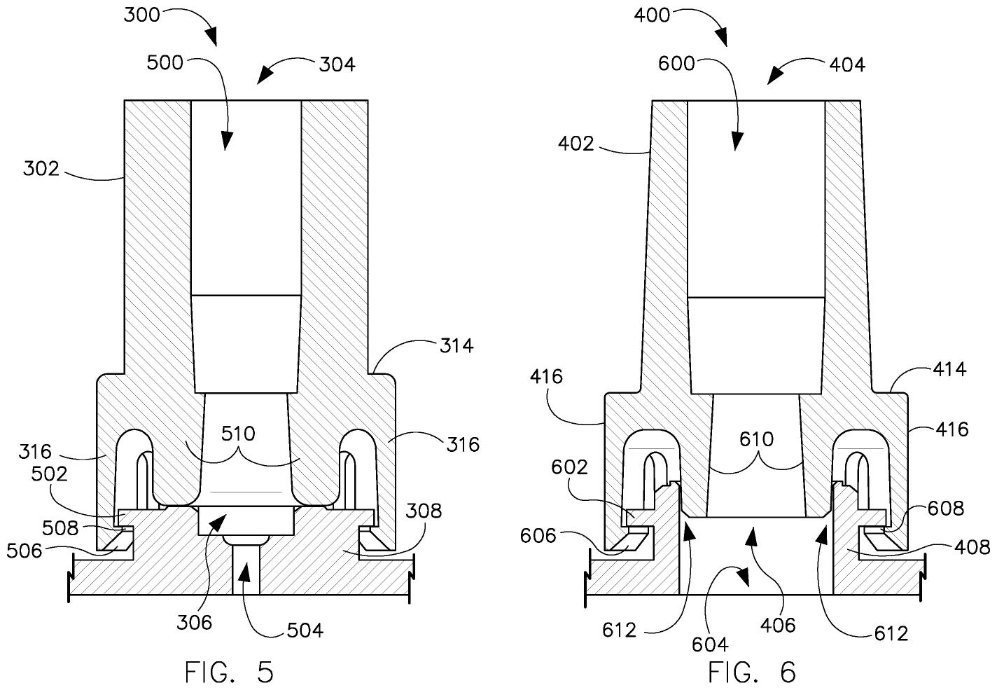

FIG. 5 is a side cross-sectional view of the example connector 300. In the example shown, the connector 300 includes a passageway 500 therethrough to provide fluid communication between the first end 304 and the second end 306. This provides fluid communication into/out of the port 308 through the connector 300. As shown, the latching projections 316 couples with a flange 502 on the port 308. In this example, the latching projections 316 provide an interference fit with the port 308. The latching projections 316 create a leaky connection with the port 308 so that sterilant fluid 132 is introduced into the internal passage 504 of port 308 and leaked onto external surfaces of the port 308. As shown, the connection between the connector 300 and the port 308 is unsealed so that sterilant fluid 132 flowing through the passageway 500 leaks out of the second end 306 onto the external surface of the port 308.

In the example shown, the latching projections 316 include a cam surface 506 and a lip 508. To couple the connector 300 with the port 308, the second end 306 of the connector 300 is aligned with the port 308. The latching projections 316 are pushed over the flange 502 of the port 308. With this action, the cam surface 506 rides on the flange 502 thereby moving the latching projections 316 outward. Upon reaching the lip 508, the latching projections 316 will resiliently move inward to form an interference coupling with the port 308. In the example shown, the connector 300 includes a wall 510 that is generally concentric with the latching projections 316. As shown, the wall 510 abuts with the external surface of the port 308. In this example, the wall 510 also acts as a stop to prevent further forward movement after engagement of the latching projections 316. Since the wall 510 is unsealed, there is a gap for sterilant fluid to leak out onto the external surface of the port 308. In some cases, the wall 510 may include micro-channels for leaking sterilant fluid onto the external surfaces of the port 308. Likewise, in this embodiment, there is a gap between the lip 508 and the flange 502 of the port 308 for sterilant fluid to flow. Accordingly, sterilant fluid flowing through the passageway 500 will both flow into the internal passage 504 of the port 308 and leak onto the external surface of the port 308 through gaps in the wall 510 and the lip 508. In some cases, the connector 300 will move during flow of sterilant fluid 132 due to gap(s) between the connector 300 and the port 308; contact between the surfaces of the connector 300 and the port 308 during movement further facilitates to decontaminate external surfaces of the port 308.

FIG. 6 is a side cross-sectional view of the example connector 400. In the example shown, the connector 400 includes a passageway 600 therethrough to provide fluid communication between the first end 404 and the second end 406. This provides fluid communication into/out of the port 408 through the connector 400. As shown, the latching projections 416 couples with a flange 602 on the port 408. In this example, the latching projections 416 provide an interference fit with the port 408. The latching projections 416 create a leaky connection with the port 408 so that sterilant fluid 132 is introduced into the internal passage 604 of port 408 and leaked onto external surfaces of the port 408. As shown, the connection between the connector 400 and the port 408 is unsealed so that sterilant fluid 132 flowing through the passageway 600 leaks out of the second end 406 onto the external surface of the port 408.

In the example shown, the latching projections 416 include a cam surface 606 and a lip 608. To couple the connector 400 with the port 408, the second end 406 of the connector 400 is aligned with the port 408. The latching projections 416 are pushed over the flange 602 of the port 408. With this action, the cam surface 606 rides on the flange 602 thereby moving the latching projections 416 outward. Upon reaching the lip 608, the latching projections 416 will resiliently move inward to form an interference coupling with the port 408. In the example shown, the connector 400 includes a wall 610 that is generally concentric with the latching projections 416. As shown, the wall 610 is received within the passage 604 of the port 408; however, the wall 610 is dimensioned so there is a gap 612 between the passageway 604 and the wall 610 for sterilant fluid to flow outwardly onto external surfaces of the port 408. Since the wall 610 is unsealed, the gap 612 provides a path for sterilant fluid to leak out onto the external surface of the port 308. Likewise, in this embodiment, there is a gap between the lip 608 and the flange 602 of the port 408 for sterilant fluid to flow. Accordingly, sterilant fluid flowing through the passageway 600 will both flow into the internal passage 604 of the port 408 and leak onto the external surface of the port 408 through gaps in the wall 610 and the lip 608. In some cases, the connector 400 will move during flow of sterilant fluid 132 due to gap(s) between the connector 400 and the port 408; contact between the surfaces of the connector 400 and the port 408 during movement further facilitates to decontaminate external surfaces of the port 408.

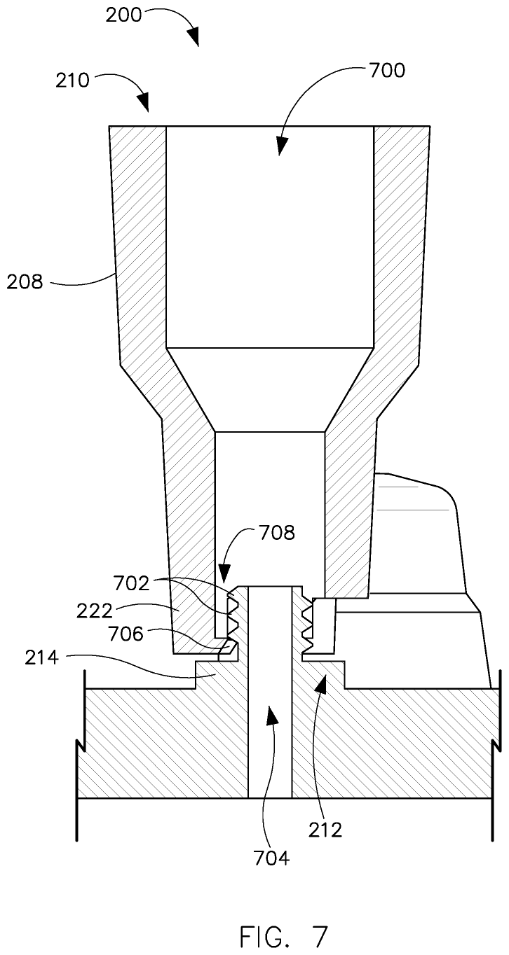

FIG. 7 is a side cross-sectional view of the example connector 200. In the example shown, the connector 200 includes a passageway 700 therethrough to provide fluid communication between the first end 210 and the second end 212. This provides fluid communication into/out of the port 214 through the connector 200. As shown, the latching projections 222 couples with threads 702 on the port 214. In this example, the latching projections 222 provide an interference fit with the port 214. The latching projections 222 create a leaky connection with the port 214 so that sterilant fluid 132 is introduced into the internal passage 704 of port 214 and leaked onto external surfaces of the port 214. As shown, the connection between the connector 200 and the port 214 is unsealed so that sterilant fluid 132 flowing through the passageway 700 leaks out of the second end 212 onto the external surface of the port 214.

In the example shown, the latching projections 222 include a lip 706 for engaging threads 702 of the port 214. To couple the connector 200 with the port 214, the second end 212 of the connector 200 is aligned with the port 214. The lip 706 is threaded onto the threads 702 of the port 214. In the example shown, there is a gap 708 into which sterilant fluid 132 may flow, which introduces sterilant fluid 132 onto external surfaces of the port 214. Accordingly, sterilant fluid flowing through the passageway 700 will both flow into the internal passage 704 of the port 214 and leak onto the external surface of the port 214 through gap 708. Since the threaded connection is unsealed, sterilant fluid may leak out onto external surfaces of the port 214.

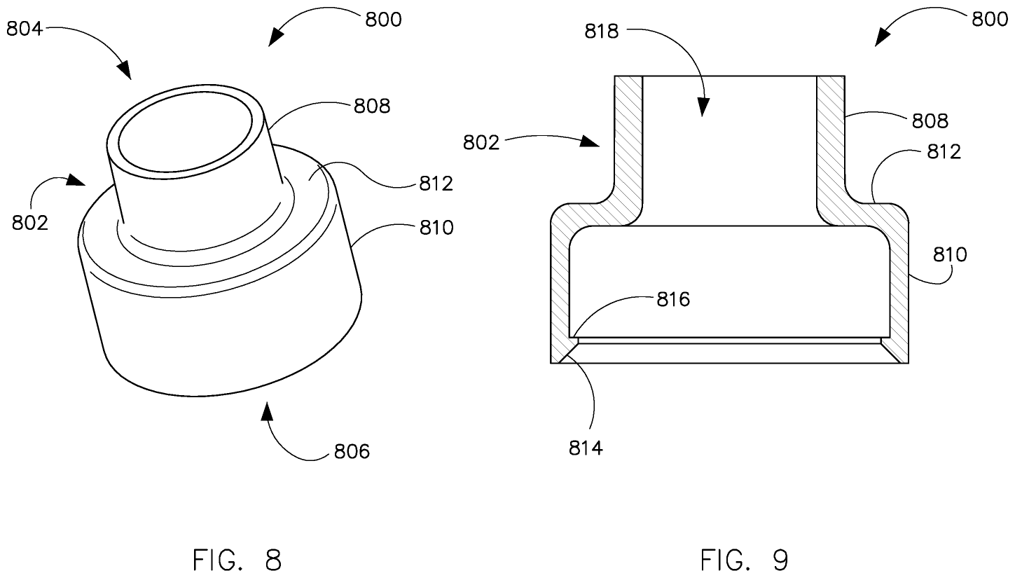

FIGS. 8 and 9 illustrate a connector 800 according to a fourth embodiment in which the connector 800 is formed from a porous material. In the embodiment shown in FIG. 8, the connector 800 includes a body 802 with a first end 804 and an opposing second end 806. In the embodiment shown, the first end 804 is configured to be connected with a sterilant fluid delivery device, such as a hookup system, to push or pull sterilant fluid into/out of the lumen device 120. The second end 806 is configured to establish a “leaky” connection with a second port of the lumen device 120. As shown, the body 802 of the connector 800 includes a first section 808 extending from the first end 804 to a second section 810. In the embodiment shown, the second section 810 has a larger diameter than the first section 808, with a shoulder 812 transitioning between the sections 808, 810.

It should be understood that the connectors and ports described herein can have different corresponding shapes so that the appropriate connector and corresponding port match, whereby they are designed so that they cannot be connected incorrectly to one another.

As best shown in FIG. 9, the second section 810 terminates with a cam surface 814 and a lip 816. The cam surface 814 and lip 816 are configured to couple with a flange of a port on the lumen device 120. For example, the cam surface 814 could ride on the flange of the port, thereby moving the second section 810 outwardly until the lip 816 latches on the flange of the port. In some embodiments, this creates a leaky connection between the connector 800 and the port so that sterilant fluid is introduced through a passageway 818 in the connector 800 into the lumen device 120 and onto external surfaces of the port. Additionally, as discussed above, at least a portion of the connector 800 is formed from a porous material; accordingly, sterilant fluid is able to flow through the first and/or second section 808, 810 onto the port of the lumen device 120.

The connector 800 could be formed from a variety of suitable porous materials that provide a balance of flow between the interior of the lumen device 120 and the external surfaces of the port. In some embodiments, the connector 800 could be formed, at least in part, from one or more of the products by Porex Corporation of Fairburn, Ga., USA:

Porex Product Number 4900, made from polyethylene, 0.0625 inch thickness, 15-45 μm pore size

Porex Product Number 4901, made from polyethylene, 0.125 inch thickness, 15-45 μm pore size

Porex Product Number 4902, made from polyethylene, 0.25 inch thickness, 15-45 μm pore size

Porex Product Number 4903, made from polyethylene, 0.0625 inch thickness, 50-90 μm pore size

Porex Product Number 4904, made from polyethylene, 0.125 inch thickness, 50-90 μm pore size

Porex Product Number 4906, made from polyethylene, 0.125 inch thickness, 90-130 μm pore size

Porex Product Number 4907, made from polyethylene, 0.25 inch thickness, 90-160 μm pore size

Porex Product Number 102074, made from polypropylene, 0.125 inch thickness, 80-155 μm pore size

Porex BM60, made from polytetrafluoroethylene, 2 mm inch thickness, approximately 3 μm pore size

These materials are provided merely for example purposes and other suitable porous materials could be used to form connector 800. These example materials have been found to be suitable to introduce sterilant fluid onto external surfaces of the port and into the lumen device 120, but other suitable porous materials could be used in this embodiment.

### Examples

Illustrative examples of the method and system disclosed herein are provided below. An embodiment of the method and system may include any one or more, and any combination of, the examples described below.

Example 1 is a decontamination system for a lumen device. The decontamination system comprises a lumen device container and a sterilant fluid delivery device. The lumen device container defines a lumen device receiving area that includes a fluid connector in fluid communication with the lumen device receiving area. The sterilant fluid delivery device is configured to be in fluid communication with the fluid connector. The fluid connector is configured to form a loose connection with a port of a lumen device to deliver sterilant fluid into the port and leak sterilant fluid onto an external surface of the port.

In Example 2, the subject matter of Example 1 is further configured such that the loose connection includes a gap between the fluid connector and the port through which sterilant fluid is configured to flow.

In Example 3, the subject matter of Example 1 is further configured such that the fluid connector is formed from a non-porous material.

In Example 4, the subject matter of Example 3 is further configured such that the fluid connector is formed from a plastic material.

In Example 5, the subject matter of Example 1 is further configured such that the fluid connector includes a coupling portion with a plurality of latching projections configured to form the loose connection with the port.

In Example 6, the subject matter of Example 5 is further configured such that at least a portion of the plurality of latching projections are arranged to create a gap between the latching projections and port through which sterilant fluid is configured to flow onto the external surface of the port.

In Example 7, the subject matter of Example 6 is further configured such that the plurality of latching projections are arranged in a ring-shape.

In Example 8, the subject matter of Example 7 is further configured such that the fluid connector includes an outlet port configured to engage the port of the lumen device.

In Example 9, the subject matter of Example 8 is further configured such that the outlet port is substantially concentric with the plurality of latching projections.

In Example 10, the subject matter of Example 91 is further configured such that at least a portion of the gap extends between the outlet port and the plurality of latching projections.

In Example 11, the subject matter of Example 1 is further configured such that the fluid connector is formed from a porous material.

In Example 12, the subject matter of Example 11 is further configured such that the fluid connector has a pore size between approximately 15-160 micrometers.

Example 13 is a connector for connecting a lumen device to a source of sterilant fluid in an endoscope reprocessor. The connection comprises a connector body including an inlet configured to be in fluid communication with a sterilant fluid delivery device and an outlet configured to be in fluid communication with a port of a lumen device. The connector body is configured to form a loose connection with the port of the lumen device to deliver sterilant fluid into the port and leak sterilant fluid onto an external surface of the port.

In Example 14, the subject matter of Example 13 is further configured such that the connector body is formed from a non-porous material.

In Example 15, the subject matter of Example 14 is further configured such that the loose connection is configured to create a gap between the connector body and the port through which sterilant fluid is configured to flow.

In Example 16, the subject matter of Example 15 is further configured such that the connector body includes at least one annular projection configured to couple the connector body with the port.

In Example 17, the subject matter of Example 16 is further configured such that at least one annular projection includes a latch portion.

Example 18 is a method of reprocessing an endoscope. The method includes the step of coupling a fluid connector of an endoscope reprocessor with a port of a lumen device. The fluid connector is configured to form a loose connection with the port such that the loose connection includes one or more gaps between the fluid connector and the port through which sterilant fluid is configured to flow. The method includes the step of introducing sterilant fluid into the port through the fluid connector. The sterilant fluid is introduced onto the external surface of the port through the gap between the fluid connector and the port.

In Example 19, the subject matter of Example 18 is further configured such that the fluid connector is formed from a non-porous material.

In Example 20, the subject matter of Example 18 is further configured such that the fluid connector includes a coupling portion with a plurality of latching projections configured to form the loose connection with the port.

Although the present disclosure has been described with reference to particular means, materials and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A decontamination system for a lumen device, the decontamination system comprising:
a lumen device container defining a lumen device receiving area, wherein lumen device container includes a fluid connector in fluid communication with the lumen device receiving area;
a sterilant fluid delivery device configured to be in fluid communication with the fluid connector; and
wherein the fluid connector is configured to form a loose connection with a port of a lumen device to deliver sterilant fluid into the port and leak sterilant fluid onto an external surface of the port.

2. The decontamination system of claim 1, wherein the loose connection includes a gap between the fluid connector and the port through which sterilant fluid is configured to flow.

3. The decontamination system of claim 1, wherein the fluid connector is formed from a non-porous material.

4. The decontamination system of claim 3, wherein the fluid connector is formed from a plastic material.

5. The decontamination system of claim 3, wherein the fluid connector includes a coupling portion with a plurality of latching projections configured to form the loose connection with the port.

6. The decontamination system of claim 5, wherein at least a portion of the plurality of latching projections are arranged to create a gap between the latching projections and port through which sterilant fluid is configured to flow onto the external surface of the port.

7. The decontamination system of claim 6, wherein the plurality of latching projections are arranged in a ring-shape.

8. The decontamination system of claim 7, wherein the fluid connector includes an outlet port configured to engage the port of the lumen device.

9. The decontamination system of claim 8, wherein the outlet port is substantially concentric with the plurality of latching projections.

10. The decontamination system of claim 9, wherein at least a portion of the gap extends between the outlet port and the plurality of latching projections.

11. The connector of claim 1, wherein the fluid connector is formed from a porous material.

12. The connector of claim 11, wherein the fluid connector has a pore size between approximately 15-160 micrometers.

13. A connector for connecting a lumen device to a source of sterilant fluid in an endoscope reprocessor, the connector comprising:
a connector body including an inlet configured to be in fluid communication with a sterilant fluid delivery device and an outlet configured to be in fluid communication with a port of a lumen device;
wherein the connector body is configured to form a loose connection with the port of the lumen device to deliver sterilant fluid into the port and leak sterilant fluid onto an external surface of the port.

14. The connector of claim 13, wherein the connector body is formed from a non-porous material.

15. The connector of claim 14, wherein the loose connection is configured to create a gap between the connector body and the port through which sterilant fluid is configured to flow.

16. The connector of claim 15, wherein the connector body includes at least one annular projection configured to couple the connector body with the port.

17. The connector of claim 16, wherein the at least one annular projection includes a latch portion.

18. A method of reprocessing an endoscope, the method comprising the steps of:
coupling a fluid connector of an endoscope reprocessor with a port of a lumen device, wherein the fluid connector is configured to form a loose connection with the port, wherein the loose connection includes one or more gaps between the fluid connector and the port through which sterilant fluid is configured to flow;
introducing sterilant fluid into the port through the fluid connector; and
introducing sterilant fluid onto the external surface of the port through the gap between the fluid connector and the port.

19. The method of claim 18, wherein the fluid connector is formed from a non-porous material.

20. The method of claim 18, wherein the fluid connector includes a coupling portion with a plurality of latching projections configured to form the loose connection with the port.
